(19) United States
(12) Patent Application Publication
(10) Pub. No.: US 2012/0158066 A1
(43) Pub. Date: Jun. 21, 2012

(54) ADJUSTABLE CERVICAL PLATE

(75) Inventor: Andrew Freese, Chester Springs, PA (US)

(73) Assignee: Camber Spine Technologies, LLC

(21) Appl. No.: 12/973,685

(22) Filed: Dec. 20, 2010

(51) Int. Cl. A61B 17/88; A61B 17/70

(52) U.S. Cl. 606/279; 606/246

(57) ABSTRACT

Modified anterior or posterior fusion plates and interbody fusion devices have been developed that contain slots to allow the use of pins to position and to guide the placement of the plate while the screws are being secured, reducing the likelihood of improper placement and localization of the plate and screws. This reduces the amount of time required for the operative procedure, and prevents inadvertent introduction of the screws for the plate into the former pin holes. This system allows the surgeon to carefully identify the geometry of the vertebral bodies at the outset of the case, when there is no significant bleeding, and when the field is clearly visualized, to determine the placement of the distracting pins. Then, the careful placement of the distracting pins serves as the guide and basis for placement of the plate and screws.

### TECHNICAL FIELD

This invention relates to appliances used in the stabilization and fusion of spinal vertebrae during and after spine surgery, and more specifically, relates to systems and methods for using a fusion plate for stabilizing vertebrae as part of a corpectomy or discectomy procedure to allow bone growth to occur.

### BACKGROUND OF THE INVENTION

Fusion plates are used to immobilize and fuse adjacent spinal vertebrae following a discectomy (spinal disc removal) or for immobilizing the area surrounding a corpectomy (removal of an entire vertebral body). These procedures create a gap in the spine from the removed disc or vertebral body. The gap typically is closed by inserting a bone graft, usually from a cadaver. The adjacent vertebrae surrounding the discectomy or corpectomy site are then immobilized by attaching a fusion plate, usually on the anterior side of the spine, so that the vertebrae fuse to the bone graft, forming an entire fused section of the spine. Fusing of vertebrae to the bone graft requires that the vertebrae remain immobile.

Presently, in performing a discectomy or corpectomy, a device called a “distractor” is used to spread the adjacent vertebrae so that the disc or vertebral body of interest can be removed. In use, a pair of distractor pins, which are essentially screws having a head for engaging with the distractor, are screwed into the vertebrae adjacent to the discectomy or corpectomy site. One pin is placed in the upper vertebra, and a second pin is placed in the lower vertebra, both vertebrae being directly adjacent to the discectomy or corpectomy site. The distractor tool is then coupled to the pins on the upper and lower vertebrae, above and below the site, and the vertebrae are then mechanically spread apart, for aiding in the removal of any remaining portion of the deteriorated disc or vertebral body, and also to create a gap for placing a bone graft. Once the bone graft is placed, the distractor is removed. The distractor pins are then removed from the spine and a fusion plate is placed in a position keeping the adjacent upper and lower vertebrae as well as the bone graft immobilized. The plate is screwed into the upper and lower vertebrae. Examples of fusion plates presently existing in the art are those produced by EBI Biomet, Inc., Dupuy AcroMed, Inc., and Synthese, Inc, to name a few.

Cervical fixation devices are commonly used to stabilize the cervical spine and promote successful bony fusion. Cervical fixation devices, including plate/screw devices, have been described for both the anterior and posterior application to the cervical spine. In spite of this, however, the available cervical plating systems have certain features that limit their usefulness in cervical spine reconstructive surgery. First, application of the available cervical plates requires a number of complex steps during surgery. Often, due to cumbersome instrumentation, application of a cervical plate may necessitate unnecessary steps to correctly size and apply the plate to the spine. Second, cervical plating systems often prevent accurate visualization of the bone graft/vertebra junction thus obscuring accurate placement of the cervical plate. Third, cervical plating systems often do not allow for compression of the bone graft, a step that is important in promoting bony fusion. Fourth, cervical plating systems generally use a cumbersome mechanism with several steps to prevent back out of the screws from the plate. Fifth, many cervical systems do not allow variability in placement of cervical screws and thus are not adaptable to anatomic variations in the cervical vertebra.

Moreover, current anterior cervical plating systems require the prior removal of the distractor pins placed in the vertebral bodies for distraction of the disc space during surgery and placement of the graft. This means that precise information about the localization of the vertebral bodies, their geometry and dimensions, which is garnered at the outset of the case during the placement of the pins, is lost. After the pins are removed at the end of the case, and frequently bone wax introduced to prevent bleeding from the pin sites, a plate is selected and introduced, typically by placing screws into the vertebral bodies above and below the fusion mass (graft). Error is introduced during this somewhat clumsy process, and the holes where the pins had been placed have to be avoided by the screws, for fear that entering the holes might not allow optimum securing of the screws.

Therefore, a need exists for a fusion plate system and method which allows a section of spine to be precisely compressed following a corpectomy or discectomy, so that sufficient and optimal immobilization and spinal fusion can occur. Additionally, a need exists for a fusion plate system and method which allows a fusion plate to be centered properly upon a spine.

Still further, there is a need for a fusion plate system that allows the plate to be positioned without first removing the distractor pins.

### SUMMARY OF THE INVENTION

Modified anterior or posterior fusion plates and interbody fusion devices have been developed that contain slots to allow the use of pins to position and to guide the placement of the plate while the screws are being secured, reducing the likelihood of improper placement and localization of the plate and screws. This reduces the amount of time required for the operative procedure, and prevents inadvertent introduction of the screws for the plate into the former pin holes. This system allows the surgeon to carefully identify the geometry of the vertebral bodies at the outset of the case, when there is no significant bleeding, and when the field is clearly visualized, to determine the placement of the distracting pins. Then, the careful placement of the distracting pins serves as the guide and basis for placement of the plate and screws. The principles are demonstrated in the visual images below, which show successful reduction to practice of this concept. Furthermore, modifications of this design are also illustrated to allow optimal placement of the plate and screws, permitting almost limitless configurations needed surgically.

### BRIEF DESCRIPTION OF THE DRAWINGS

FIG. 1 is a prospective view of a cervical spinal fusion plate.

FIG. 2A is a perspective view of a cervical spinal fusion plate with pins inserted; FIG. 2B is a side perspective view of the plate with pins inserted and FIG. 2C is a cross-sectional view of the plate with pins inserted.

FIGS. 3A-3D are views of the cervical spinal fusion plate with enclosed slots for placement of the distractor pins. FIG. 3A is a perspective view of the plate; FIG. 3B is a top view of the plate showing the top of the inserted pins; FIG. 3C is a cross-sectional view of the plate and pins; and FIG. 3D is a prospective view of the plate with pins inserted.

FIG. 4 is a perspective view of a distractor pin.

### DETAILED DESCRIPTION OF THE INVENTION

### I. Plate and Pin System

Placement of a distraction pin early in the course of an anterior cervical spine operation is commonly done to allow accurate distraction of the disc space and promote a thorough decompression of the spinal cord and nerve roots. The distraction pins are generally placed in the midline of the vertebral body at a 90 degree angle to the vertebra. This position is ideal for reference throughout the operation. Because the distraction pins are placed early in the operation, prior to distortion of the anatomy by surgical dissection, they represent the most accurate reference site for placement of an anterior cervical plate.

An anterior cervical plating system has been designed to reference the size and location of the cervical plate from the distraction pins in the vertebral body. The distraction pins serve several functions besides vertebral distraction during decompression, allowing correct sizing of the plate, compression of the bone graft during plating and provisionally securing the plate during drilling of the screw holes and application of the screws. In this way, the surgeon can move immediately from the decompression to grafting and plating without the need of removal of the distraction pins. The distraction pins provide rapid and accurate information on the size of the plate and allow graft compression and provisional plate fixation. A series of complex surgical steps is simplified into a simple exercise using the distraction pins in this way.

The plate incorporates a series of other features designed to promote ease of application and flexibility of use during anterior cervical plating. These features included “windows” in the plate to allow visualization of the bone graft, variable angled screws, a simple screw locking mechanism (requiring no additional steps), a sliding segment to allow fixation of intervening vertebral bodies and a reference guide for drilling screw holes that allows accurate placement of the screws based on the position of the distraction pins.

Distraction pins are inserted into the vertebral body, but contain a mechanism to capture the anterior cervical plate. The pins are designed with a “stop” (expanded portion to prevent over drilling of the pin) and a second “lip” under which the plate is captured and provisionally held during plating.

The distractor pins currently in use have a broad flange that prevents their removal if a plate is placed on top of the flange. This problem is obviated by the design of the pins described herein to allow placement of the plate with the distractor pin positioned within the veterbral body but still capable of being easily removed.

The anterior cervical plate has an adaptation at each end to allow the ends of the plate to be secured by the distraction pins. The adaptation is an indention in the plate at each end with a thin portion that can be captured under the “lip” of the distraction pin. Alternatively, the plate can have holes in each end allowing it to slide over the distraction pins.

The anterior cervical plate also contains “windows” over the edges of the bone graft so that the graft/vertebra junction can be visualized accurately during plating.

The anterior cervical plate has a mechanism to allow variable angulation of the screw while allowing the screws to be attached to the plate without the need for separate steps. This consists of “expandable bushings” that can rotate in desired directions within the plate. These bushings are internally threaded to allow screws to be placed through the bushings. The screws can thus be angled in a desired direction. The screws are designed with a screw head that expands the bushing during terminal seating of the screw so that the bushing is expanded and fixed in position to the hole in the plate. In addition, the screw head has a shape that prevents it from loosening or backing out once it is well seated into the expandable bushing.

The anterior cervical plate may also contain a sliding segment or segments to allow fixation of intervening vertebral bodies with screws. This consists of cross member(s) that can be attached to the lateral structures of the plate. These cross member(s) have screw holes with expandable bushings to allow the placement of vertebral screws. The cross members can be adjusted in position towards the top of bottom of the plate such that they are optimally positioned over an intervening vertebral body.

The slots allow for positioning of the plates after placement of the pins but prior to removal. The pins may include a sleeve to enhance retention of the pin in the slot during placement. Alternatively, the slot may be ridged or beveled to retain the pin within the slot during placement.

The drill guide for screw placement is designed so that accurate angulation of the drill holes can be referenced from the distraction pins. The pins can be used to introduce hemostatic agents, such as bone wax, to prevent bleeding once they are removed.

These plates, pins and drill guides, and method for installing this system upon a patient's spine, overcome the drawbacks of the prior art by allowing a desired level of compression to be applied to the adjacent vertebrae surrounding the site of a corpectomy or discectomy, prior to, and during, the anchoring of the fusion plate. Furthermore, the fusion plate system and method results in the fusion plate being properly centered upon a patient's spine, so that an aesthetically pleasing, as well as functional, surgical result is achieved.

The system and method uses distractor pins to properly guide the fusion plate to a centered positioning upon a patient's spine. Once guided onto the spine, the fusion plate is anchored with bone screws. The distractor pins are centered on the spine using anatomical landmarks such as the longis colli muscles or uncinate processes.

Plates and pins are preferably made of surgical stainless steel or titanium. Most other materials lack sufficient strength under stress.

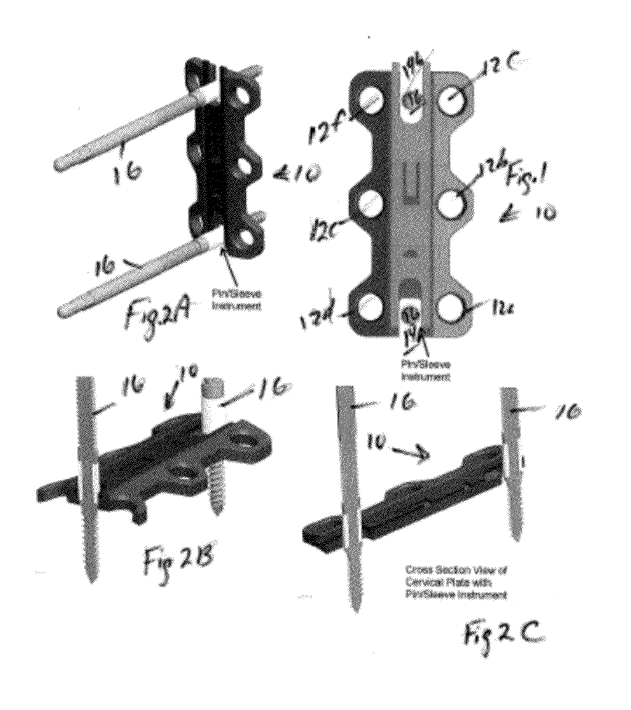

As shown in FIG. 1, the plate 10 has holes 12a, 12b, 12c, 12d, 12e, and 12f, for screws to secure the plate 10 to the vertebrae. The plate has slots 14a and 14b for placement of distractor pins 16, shown in FIGS. 2A, 2B and 2C. The slots 14a, 14b allow placement, manipulation, and removal of pins after insertion of screws through holes 12a-1f.

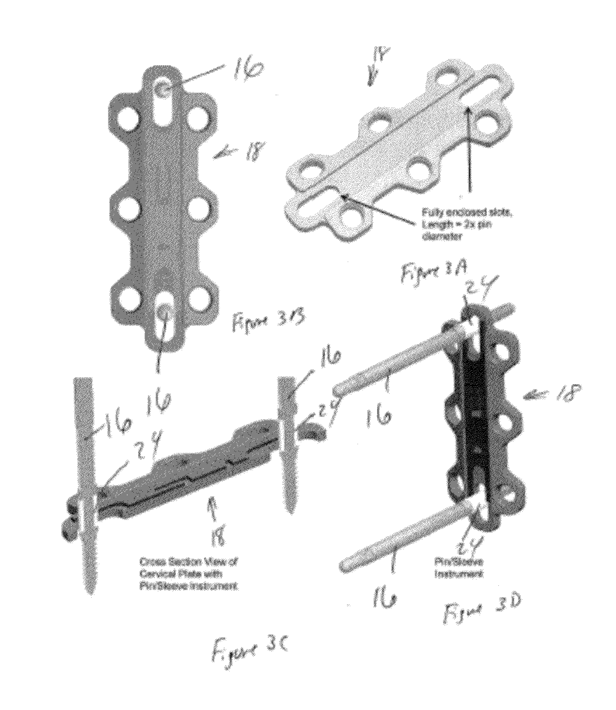

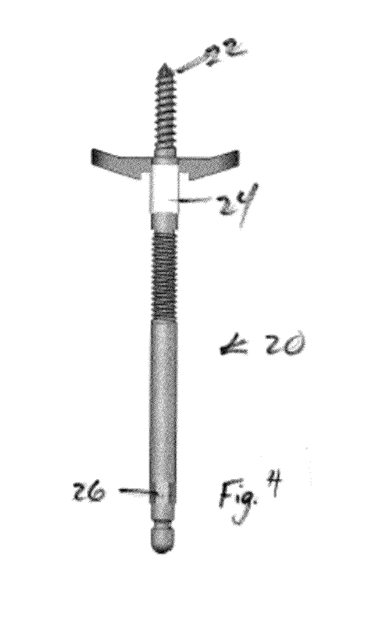

Another embodiment of the plates is shown in FIGS. 3A-3D. These plates 18 have fully enclosed slots 19 for pin placement.

The plates will typically be provided in several sizes, typically ranging from 20 to 70 mm. These are used to accommodate different sized bones.

A pin 20 is shown in FIG. 4. The pin has a threaded bone entry end 22, a sleeve 24, and a multi-faceted tool fitting 26. The pins are used to distract the discs and to place the plate. These guide the plate as well as secure the plate until the screws are positioned. Unlike conventional plates, the pins are not removed until after the plates are secured with the screws. The pins allow compression across the plate before attaching the screws, thereby enhancing the likelihood of a successful fusion. The sleeve, typically formed of an elastomer or polymeric material such as polypropylene, or formed of a ridged metal as an integral part of the slot, holds the pin in place within the slot in the plate as it is screwed in. This makes it possible to position the pins using only one hand, instead of requiring one hand to turn the pin and one hand to hold the pin in the plate.

Unique features of the pins include that they can be pushed apart, the plates put into position, then used to compress the opening prior to securing the screws. In contrast, current systems require removal of the pins prior to placement of the plate and positioning of the screws. This is achieved through the use of the slots at each end of the plates. With conventional plates, some of the compression is lost during the interval between placement of the screws and removal of the pins. This system also allows more bone graft to be used. Typically, the hole left by removal of the pins will be filled with bone wax or other sealant to prevent bleeding.

### II. Positioning of the Pins and Plates

Plates, screws and pins are typically provided in a sterile kit or kits. The kit may also include tool(s) for placement of the pins and/or screws such as drills, taps, and drivers.

The physician will select the appropriately-sized plate for fixation to the vertebral bodies. A properly sized plate will bridge the affected segments without overhanging into adjacent disc space.

The pins are inserted into the placement tool and screwed into the vertebral bodies. The plate is then positioned with the pins holding the plate in place. Screw holes are drilled and then the screws inserted. The screws may be self-drilling or self-tapping screws, with cutting or blunt tips. The pins are then removed after the screws are secured.

In the preferred embodiment, the method of using the plates and pins described herein is as follows:

Place distractor pins in vertebrae;
Use distractor system used to push pins apart (i.e., distract) to expand the disc space;
Remove disc;
Identify appropriate sized bone graft or cage;
Place bone graft or cage into the disc space;
Remove distractor system but not pins;
Select appropriately sized plate that is defined by the space between the pins;
Place the plate into position with the pins;
Apply distractor system to compress the disc space;
Secure plate with screws; and
Remove the distractor pins.

Modifications and variations of the claimed pins and plates and methods of use thereof will be obvious to those skilled in the art and are intended to come within the scope of the appended claims.

We claim:

1. A vertebral spinal or lumbar fixation plate comprising
Holes for insertion of screws to position and secure the plate on the vertebral surfaces, and
one or more slots for placement of distraction pins, wherein the slots allow positioning and removal of the pins after the plate is secured with screws.

2. The plate of claim 1 comprising slots at each end of the plate and screw holes located on both sides of the plate.

3. The plate of claim 1 wherein the slots are ridged, have bushings, or have beveled edges to stabilize the pins.

4. A distraction pin for placement of vertebral spinal or lumbar fixation plates comprising a pin having a sleeve, bushing or ridged metal region to stabilize the pin in the plate but not prevent the pin from being removed from the plate after placement.

5. The distraction pin of claim 4 comprising a polymeric sleeve or bushing.

6. The distraction pin of claim 4 comprising a threaded end to secure the pin in the vertebrae and appurtances at the other end to affix a tool for screwing in or removing the pin.

7. The distraction pin of claim 4 wherein the sleeve, bushing or ridged metal region is in the middle of the slot in the plate.

8. The distraction pin of claim 4 wherein the sleeve, bushing or ridged metal region is at one end of the pin which fits into the slot in the plate.

9. A kit comprising screws and a vertebral spinal or lumbar fixation plate comprising
Holes for insertion of screws to position and secure the plate on the vertebral surfaces, and
one or more slots for placement of distraction pins, wherein the slots allow positioning and removal of the pins after the plate is secured with screws.

10. A kit comprising distraction pins and a vertebral spinal or lumbar fixation plate comprising
Holes for insertion of screws to position and secure the plate on the vertebral surfaces, and
one or more slots for placement of distraction pins, wherein the slots allow positioning and removal of the pins after the plate is secured with screws.

11. The kit of claim 10 wherein the distraction pin comprises a pin having a sleeve, bushing or rigid metal to stabilize the pin in the plate.

12. The kit of claim 10 wherein the slots in the plate are ridged, have bushings, or have beveled edges to stabilize the pins.

13. A method for stabilizing vertebrae in an individual in need thereof comprising
providing a vertebral spinal or lumbar fixation plate comprising
Holes for insertion of screws to position and secure the plate on the vertebral surfaces, and
one or more slots for placement of distraction pins, wherein the slots allow positioning and removal of the pins after the plate is secured with screws, and
two or more distraction pins comprising a sleeve, bushing or ridged metal region to stabilize the pin in the plate but not prevent the pin from being removed from the plate after placement.

14. The method of claim 13 comprising
Placing the distractor pins in vertebrae;
Using the distractor system to expand the disc space;
Removing disc;
Placing an appropriately sized bone graft or cage into the disc space;
Removing the distractor system but not the pins;
Placing an appropriately sized plate into position with the pins;
Applying the distractor system to compress the disc space;
Securing the plate; and
Removing the distractor pins.
